(19) United States
(12) Patent Application Publication
(10) Pub. No.: US 2026/0235600 A1
(43) Pub. Date: Aug. 13, 2026

(54) MID-INFRARED OPTICAL PHOTOTHERMAL INTERFEROMETRIC MICROSCOPY SYSTEMS AND METHODS

(71) Applicant: Purdue Research Foundation, West Lafayette, IN (US)

(72) Inventors: Garth Jason Simpson, West Lafayette, IN (US); Caitlin Elizabeth Dunlap, Lafayette, IN (US); Alexander James Higgins, West Lafayette, IN (US)

(73) Assignee: Purdue Research Foundation, West Lafayette, IN (US)

(21) Appl. No.: 19/456,907

(22) Filed: Jan. 22, 2026

Related U.S. Application Data
(60) Provisional application No. 63/748,247, filed on Jan. 22, 2025.

(51) Int. Cl. G01N 33/569; G01N 1/28; G01N 21/25; G01N 21/35

(52) U.S. Cl. CPC G01N 33/56911; G01N 1/2813; G01N 21/255; G01N 21/35

(57) ABSTRACT

A method for determining absolute thickness of bacterial colonies include transferring one or more bacterial colonies to an infrared (IR) transparent substrate, shining visible light; polarizing the shone visible light, thus polarizing the visible light at a predetermined linear polarization, converting the linearly polarized light to circularly polarized light, combining IR light from a second light source with the linearly polarized light, focusing the combination of IR light and the linearly polarized light by an objective onto the IR transparent substrate with the one more bacterial colonies disposed thereon, back-reflecting probe beam light from the one or more bacterial colonies, polarizing the back-reflected probe beam light, focusing the polarized back-reflected probe beam light by a lens, receiving the polarized back-reflected probe beam light by a detector, performing optical photothermal mid-infrared (O-PTIR) measurements on the detected light while varying IR and back-reflected probe beam light power settings optimized for the substrate.

### CROSS-REFERENCE TO RELATED APPLICATIONS

The present non-provisional patent application is related to and claims the priority benefit of U.S. Provisional Patent Application Ser. No. 63/748,247, filed Jan. 22, 2025, the contents of which are hereby incorporated by reference in its entirety into the present disclosure.

### GOVERNMENT SUPPORT CLAUSE

This invention was made with government support under CHE2305178, CHE2320751, EEC2412582, IIP 1916691 awarded by the National Science Foundation and under DE-SC0022884 awarded by the Department of Energy. The government has certain rights in the invention.

### TECHNICAL FIELD

The present disclosure generally relates to instrumentation and analysis methods to aid in interpreting reflection-based optical photothermal mid-infrared (O-PTIR) microscopy measurements of samples prepared on planar substrates.

### BACKGROUND

Optical photothermal mid-infrared (O-PTIR) microscopy bridges the gap between the rich chemical specificity of mid-infrared spectroscopy and the high spatial resolution of optical microscopy. By exploiting the localized photothermal response induced by infrared absorption, O-PTIR enables label-free imaging of chemical composition at spatial resolutions far higher than those set by the diffraction limit of mid-infrared light. This capability is particularly impactful for studying heterogeneous materials, biological systems, and nanostructured surfaces, where traditional infrared absorption microscopy methods obscure the underlying microstructure. In biological systems, vibrational analysis offers insight into chemical composition, protein secondary structure, and vibrational-electronic interactions in light-activated processes such as light-harvesting and photoreception. Yet probing these processes in intact cells-let alone from specific cellular structures of complexes-remains experimentally challenging. Fully utilizing the utility of this technique relies critically on accurate interpretation of the contrast arising within the observed images. Such interpretations build directly on precise understanding of the underlying photophysical processes governing spatial resolution and image contrast.

Photothermal spectroscopy measurements performed in isotropic media are typically optimized around thermal lens optical configurations, in which large changes in detected intensity arise from subtle photothermal changes in the divergence/convergence of the probe beam. In these systems, the photothermal temperature change produces a localized modulation in refractive index, which in turn serves as a thermal lens. Appropriately positioning the thermal lens adjacent to the focus of the probe beam produces changes that impact the coherent divergence/convergence of an overlapping visible probe beam. Optimal detection of these subtle modulations is often achieved by performing measurements in “dark-field” measurement configurations, in which the unperturbed system produces a minimum in the optical transmission. Impressively low limits of detection approaching single molecule sensitivity have been achieved by thermal lens photothermal detection. Thermal lens microscopy performed in a dark-field detection geometry results in a point spread function (PSF) typically exhibiting significant axial asymmetry and non-Gaussian characteristics due to the reliance on probe beam modulation. As one prominent example, a thermal lens from a point-source positioned precisely in the focal point of the probe beam generally produces no change in divergence/convergence, resulting in a local minimum in sensitivity.

Many of these same principles from dark-field detection in isotropic media have analogous benefits in microscopy measurements, but with notable differences by nature of local structure intrinsic in samples interrogated by microscopy. In microscopically structured samples, thermal lensing is not strictly required to generate detectable photothermal perturbations in dark-field configurations. In samples already exhibiting optical scattering, modulation in the scattering cross-section can also inform on mid-infrared pump beam absorption. Under these conditions, the modulation amplitude with dark-field detection scales linearly with both the photothermal sensitivity (dictating the depth of modulation) and the total scattered intensity. Such measurements have been used in the prior art in analysis of pharmaceutically relevant powdered samples, and for sensitive detection of gold nanorods by photothermal imaging. Depending on the optical design, such instruments have the potential to inform on both coherent (thermal lens) and incoherent (optical scattering) contributions to the detected photothermal perturbation. While the PSF for the former is reasonably well-established based on thermal lensing effects, incoherent contributions to O-PTIR from modulation in the scattering cross-section have the potential to be highly dependent on the composition and physical structure of the sample, with corresponding complexities in reliable modeling of optical contrast.

In this context, samples supported on planar substrates emerge as both ubiquitous and theoretically tractable models for exploring the interplay between the coherent and incoherent contributions to the reflected and/or transmitted signal in O-PTIR microscopy. Reflection is the coherent counterpart of scattering, sharing similar dependencies on sample thermal and optical properties. Furthermore, the analysis of powders and dried samples often necessitates deposition onto solid substrates, which provide mechanical stability and infrared optical transparency.

Similarly, the relatively short penetration depth of mid-infrared radiation in aqueous media typically necessitates analysis of substrate-adherent samples in biological applications of O-PTIR microscopy. While these substrates are often practical necessities, they also introduce reflective interfaces capable of impacting the relationship between the local temperature change and the corresponding modulation in transmitted/reflected signal intensity. In such cases, the reflectivity perturbations associated with the transient temperature changes at the sample/substrate interface have the potential to produce significant modulation in the integrated fraction of light reflected or transmitted. This intensity modulation does not operate on a thermal lens mechanism and therefore does not require a dark-field detection geometry. Furthermore, the resulting PSF arising from reflectivity changes has the potential to better match the native PSF of the probe beam, unlike photothermal detection based on thermal lens effects.

In O-PTIR microscopy measurements performed on substrate-supported samples, modulation in the epi-detected or transmitted intensity can potentially arise from thermal perturbation in either the incoherent scattering or the coherent back-reflection/transmission of the probe beam. Incoherent contributions to the detected signal can be isolated experimentally using dark-field detection geometries, such as those used in prior O-PTIR measurements of powders and compacts. These measurements are distinct from dark-field geometries optimized for thermal lensing, which operate based on coherent photothermal perturbations to the divergence/convergence of the transmitted beam. In the limit of incoherent photothermal detection, the photothermal response arises from a combination of temperature-dependent changes in particle size and refractive index, which collectively perturb the scattering cross-sections of particle assemblies. In samples dominated by photothermal modulation in the incoherent scattering, one expects the O-PTIR amplitude to scale proportionally with the overall scattering cross-section. In cases for which the detected photothermal signal is dominated by the coherent optical response, optical interference effects may contribute significantly to modulation in the back-reflected beam. If present, such interactions have the potential to significantly enhance the sensitivity of O-PTIR given the established sensitivity of interferometry to subtle changes in film thickness. However, they also have the potential to impact the measured image contrast in nontrivial ways if not properly accounted.

Despite the potential advantages of detecting changes in reflectivity, little prior work is available to assist in interpreting the PSF and image contrast arising from photothermal modulation for thin biological samples supported on substrates. In work integrating differential interference contrast with photothermal microscopy within aqueous microfluidic microchannels, others considered key design criteria related to heat flow in multilayered samples, but not multi-layer optical interference effects. Other researchers have performed detailed studies comparing O-PTIR measurements of live and fixed cells for metabolic profiling, with the appearance of micro-scale contrast features in the O-PTIR measurements of fixed cells absent in corresponding bright-field images. Similarly, studies by other researchers comparing electron microscopy and IR photothermal microscopy of microplastic particulates released from teabags suggest possible modulation in intensity across the photothermal image in particulates likely to be varying smoothly by SEM. While explanations other than optical interference may well explain these prior observations, one generally does expect some degree of optical interference to be present for samples prepared on solid substrates.

In this backdrop, prior work in photothermal reflectance analysis may provide some theoretical context to aid in the interpretation of O-PTIR measurements of substrate-supported samples. Photothermal reflectance microscopy has been used to interrogate thermal conductivity within carbon nanotubes and thermal transport within 2D materials and anisotropic media. At ultrafast timescales, transient absorption imaging can differentiate between ballistic exciton transport and thermal diffusion. The primary challenge in extending this prior framework for interpreting photothermal reflectance to biological microscopy is that of scale; the prior photothermal reflectance microscopy measurements of semiconducting and metallic media were performed either for bulk or ultrathin samples only a few atomic layers thick, while the relevant length-scales of many biological assemblies are on the same order as the optical wavelength. As a consequence, optical interference effects that can justifiably be largely ignored in analysis of ultrathin films have the potential to play a much more significant role in impacting optical contrast in biological microscopy. For samples approaching the optical wavelength in thickness, optical interferometry can recover absolute thickness. Precise knowledge of the path length and the absorption cross-section supports quantitative characterization of intracellular analyte concentration within cellular microenvironments.

Therefore, there in an unmet need for a novel method and system that can aid in interpreting reflection-based optical photothermal mid-infrared (O-PTIR) microscopy measurements of samples prepared on planar substrates.

### SUMMARY

A method for determining absolute thickness of bacterial colonies includes transferring one or more bacterial colonies to an infrared (IR) transparent substrate, along a first light path, shining visible light from a first light source, polarizing the shone visible light along the first light path, thus polarizing the visible light at a predetermined linear polarization, along the first light path, converting the linearly polarized light to circularly polarized light, along the first light path, combining IR light from a second light source with the linearly polarized light, along the first light path, focusing the combination of IR light and the linearly polarized light by an objective onto the IR transparent substrate with the one more bacterial colonies disposed thereon, back-reflecting probe beam light from the one or more bacterial colonies along the first light path, polarizing the back-reflected probe beam light along a second light path, along the second light path, focusing the polarized back-reflected probe beam light by a lens, along a second light path, receiving the polarized back-reflected probe beam light by a detector, and performing optical photothermal mid-infrared (O-PTIR) measurements on the detected light while varying IR and back-reflected probe beam light power settings optimized for the substrate to thereby determine the absolute thickness of bacterial colonies.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1A is a schematic of an optical circuit illustrating design principles of an instrument diagram for reflectance-based photothermal mid-infrared (PTIR) optical system according to the present disclosure.

FIG. 1B is a depiction of axial defocusing on a detected signal, which scales with D2 (distance between objective, L1, and sample) and D3 (distance between lens, L2, and detector) on back-reflected light rays.

FIG. 1C is a graph of amplitude vs. Z-position which provides simulations and measurement of the axial point-spread function (PSF) for a beam-path design consistent with FIG. 1A and FIG. 1B, and which illustrate the relationship between the detector active area and the axial resolution in the PSF.

FIGS. 1D, 1E, 1F, and 1G, provide a summary of modeling calculations for the z-dependent PSF produced by the Cassegrain reflective objective, where the frequency-domain Gaussian widths were selected to be ⅔ of the corresponding band edges (e.g., a positive Gaussian function with a width of σ_{v̅}=2×0.78/3 and a negative Gaussian with a width of σ_{v̅}=2×0.45/3).

FIG. 1H is a block diagram that shows the optical circuit in FIG. 1A in an alternative view.

FIGS. 2A-2C provide a thin layer model for photothermal IR reflectometry, where FIG. 2A depicts a one layer model on a substrate, FIG. 2B provides a two or more layer model, and FIG. 2C provides an illustration of the collective reflection, transmission, and phase shifts driving the complex-valued reflectivity in a one-layer system.

FIGS. 3A-3F provide the results of simulations of the anticipated O-PTIR responses for a sample varying in height, acquired with a beam path similar to that depicted in FIGS. 2A-2C, where FIG. 3A illustrates the predicted sensitivity of a sample with a refractive index of 1.5 on either Si or CaF₂, for a sample with a Gaussian height distribution depicted in FIG. 3B, the O-PTIR and coherent reflectance responses for Si substrates are shown in FIGS. 3C and 3D, respectively, and the corresponding responses for a CaF₂ substrate are shown in FIGS. 3E and 3F, respectively.

FIGS. 4A-4F provide representative set of experimentally observed O-PTIR results for measurements of Synechocystis prepared on CaF₂ and silicon substrates, where raw experimental data are shown in FIGS. 4A and 4F with the maximum likelihood estimates of the experimental observables shown in FIGS. 4B and 4E, with the Gaussian sample height distribution shown in FIG. 4C and FIG. 4F.

FIGS. 5A-5F provide results for Time-averaged (DC) reflectance images obtained simultaneously with the O-PTIR micrographs, where FIG. 5A provides measured reflectance of a bacterial colony on CaF₂ substrate with the predicted incoherent contribution to reflectance, given by the intensity of the best-fit sample height profile convolved with the PSF which is provided in FIG. 5B and predictions for coherent back-reflectance which is provided in FIG. 5C, with analogous measurements and predictions for a representative colony on an Si substate are given in FIGS. 5D, 5E, and 5F.

FIG. 6 provides representative amide vibrational spectra of Synechocystis colonies on IR-transparent substrates, demonstrate the reproducibility of the trends represented in FIGS. 4A-4F.

FIGS. 7A-7H provide assessment of reproducibility of OPTIA analysis of O-PTIR data, for both CaF₂ and Si substrates, where measurements for the data shown presented herein on both silicon (FIG. 7A and FIG. 7B) and CaF₂ (FIG. 7C and FIG. 7D) substrates, the corresponding maximum likelihood fits based on OPTIA are presented in FIG. 7E, FIG. 7F, FIG. 7G, and FIG. 7H, respectively, with the best-fit values for the heights and widths provided.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 15%, within 10%, within 5%, or within 1% of a stated value or of a stated limit of a range.

In the present disclosure, the term “substantially” can allow for a degree of variability in a value or range, for example, within 85%, within 90%, within 95%, or within 99% of a stated value or of a stated limit of a range.

A novel method and system that can aid in interpreting reflection-based optical photothermal mid-infrared (O-PTIR) microscopy measurements of samples prepared on planar substrates is disclosed herein. Towards this end, a thin-film model is described herein for interpreting both the time-averaged (DC) reflectivity and the photothermally induced change in reflectivity (AC) from localized, transient heat deposition. This optical photothermal interferometry analysis (OPTIA) approach for interpreting O-PTIR microscopy measurements enables recovery of the absolute thickness of individual bacterial colonies (e.g., Synechocystis) following transfer to IR transparent substrates. In addition, large signal to noise enhancements in O-PTIR measurements were both predicted and observed for measurements performed on silicon substrates relative to calcium fluoride, attributed to a combination of a higher overall reflectivity as well as enhancements in the contributions arising from optical interference between the sample and substrate responses. Leveraging these interference effects will significantly increase the accessible information content in O-PTIR microscopy measurements and lays the foundation for additional measurement approaches specifically designed to exploit the native interferometry arising for substrate-supported samples.

The integration of optical interference considerations disclosed herein provides a predictive framework for interpreting image contrast in O-PTIR microscopy of substrate-supported samples. From the model, signal enhancements for O-PTIR microscopy on silicon substrates relative to CaF₂ were predicted and found to be in good agreement with experimental observations on measurements of microbial thin films. Furthermore, integration of interference effects in interpretation of the experimental O-PTIR micrographs enabled absolute recovery of sample thickness to within a precision of +100 nm. These results illustrate the potential advantages of leveraging interference “artefacts” in O-PTIR microscopy to recover additional information content. As one example, precise thickness measurements can in turn support recovery of absolute analyte concentration from the Beer-Lambert Law. Furthermore, the mathematical framework presented in the present disclosure supports predictions of more complex multi-layer architectures will emerge as the scope of O-PTIR microscopy continues its rapid expansion.

Instrumentation and analysis methods are presented to aid in interpreting reflection-based O-PTIR microscopy measurements of samples prepared on planar substrates. For such samples, optical interference in the back-reflected probe beam arises from the reflectivities of both the sample and substrate, with the potential to complicate conventional analysis. Furthermore, this class of samples arguably represents among the most common cases encountered in routine analysis, given the practical needs of physically supporting samples prior to optical microscopy.

A thin-film model is developed for interpreting both the time-averaged (DC) reflectivity and the photothermally induced change in reflectivity (AC) from localized, transient heat deposition.

In the present disclosure, non-limiting focus is provided on the cyanobacterium Synechocystis PCC 6803, which has emerged as an important model organism both for fundamental photosynthesis research and for bioengineering applications focused on biosynthesis of biofuels or high-value chemicals. Quantitative vibrational analysis within individual Synechocystis cells has many potential applications, from the elucidation of stimulus-response mechanisms and protein synthesis/degradation pathways to directed evolution and high-throughput screening for mutants with desirable light-harvesting or biosynthetic traits. In the specific context of photosynthetic light-harvesting, vibrational analysis is further critical for understanding vibrational-electronic mixing and energy relaxation within and between light-harvesting proteins. However, absolute changes in the absorption cross-section within a colony (produced, e.g., by genetic variation or in response to an external stimulus) depend both on the measured extinction and the optical path length through the bacterial colony, which is typically not known experimentally. Independent determination of path length in combination with protein concentration by infrared spectroscopy can support precisely such quantitative characterization, provided the path length through the sample is known.

To address both the benefits of accessing absolute thickness determination and the challenges in interpreting image contrast in O-PTIR microscopy of substrate-supported samples, the present disclosure centers on dissecting the dominant contributions to the photothermally modulated back-reflected intensity. To support this analysis, analytically tractable models are proposed for describing the dominant elements contributing to coherent reflectivity and transmissivity changes induced upon photothermal modulation for samples measured at the surface of a supporting substrate. The predictions of these models are compared to experimental observations for thin films of Synechocystis bacterial colonies transferred to commonly employed IR transparent substrates. The proposed thin-film models aim to provide a framework for the quantitative interpretation of optical contrast in photothermal mid-infrared microscopy measurements of substrate-supported samples.

Wild-type (WT) and antenna-truncated (AT) mutant Synechocystis PCC 6803 colonies were grown on BG11 agar plates at 28° C. under constant 30 uE light. The AT mutant was prepared through recombinant transformation removing of both allophycocyanin (ΔApcAC, erythromycin resistance cassette) and the anchor protein (ΔApcE, spectinomycin resistance cassette), that connects the antenna proteins allophycocyanin and phycocyanin to the thylakoid membrane. Glucose (10 mM), TES buffer (10 mM, pH 8.2), and sodium bicarbonate (5 mM) were added to both plates to allow for hetero-autotrophic growth. WT Synechocystis was grown without antibiotics, while erythromycin (25 μg/mL) was added to the ΔApcAC/ΔApcE plate to suppress residual unmodified bacteria.

Calcium fluoride (CaF₂, UQC Optics) and silicon (WaferPro, 2″, P type, B-doped, 1-5 ohm-cm resistivity) slides were placed in direct contact with the agar plate, applying gentle pressure to transfer microbial colonies onto the slide surface. Following this imprinting process, the slides were carefully removed from the agar plate and positioned within the sample chamber of the O-PTIR microscope for further analysis.

O-PTIR measurements were performed with varying IR and probe power settings optimized for each substrate. For CaF₂ substrates, IR power was optimized to be 22% (about 12 mW, Daylight Solutions QCL, 100 kHz pulse repetition rate, 100 ns pulse width), while the probe power was maintained at 20% (about 50 mW, ThorLabs, CW, 532 nm). In contrast, for silicon substrates, the probe power was reduced to 7.4% (corresponding to about 20 mW) to account for the increased back-reflectance. All measurements were conducted within the spectral range of 1450 cm⁻¹ to 1800 cm⁻¹, targeting the amide peak region characteristic of cyanobacteria.

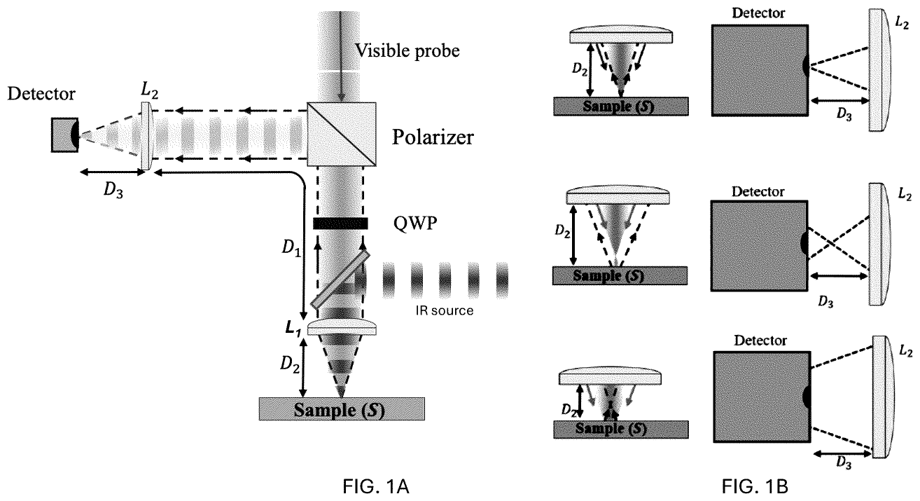

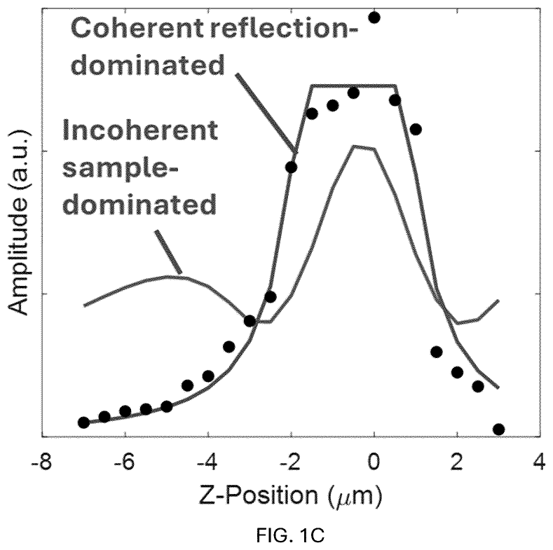

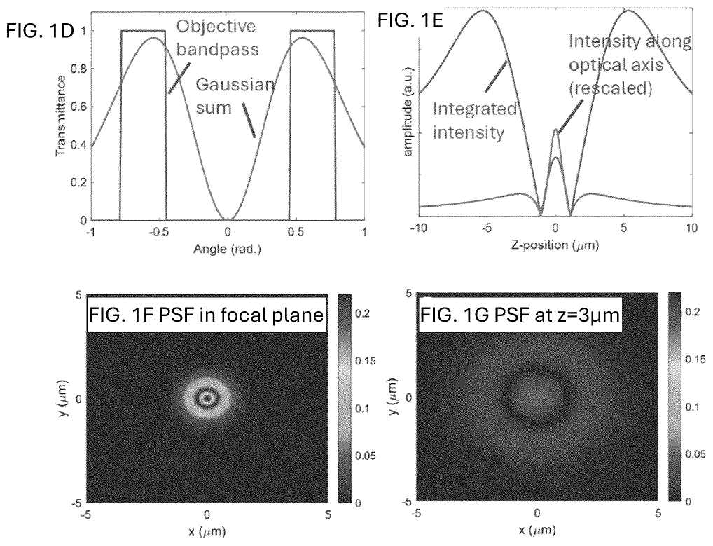

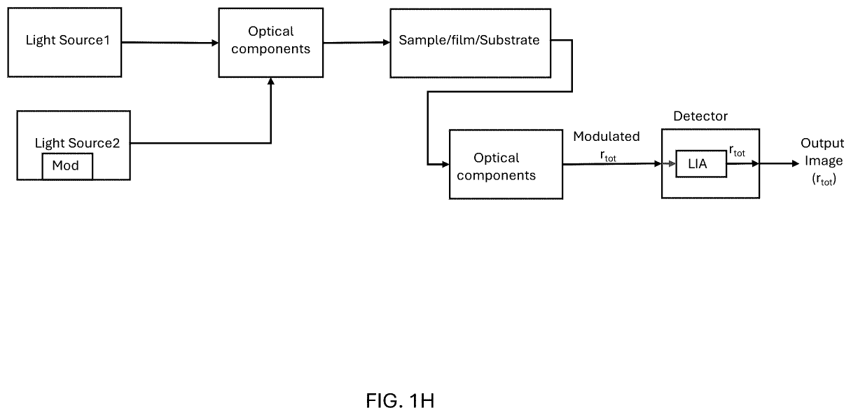

One representative design is depicted in FIG. 1A, which is a schematic illustrating design principles of an instrument diagram for reflectance-based PTIR optical system according to the present disclosure. In particular, FIG. 1A is a schematic of representative beam-path for back-reflection detection assuming thin lenses L1 and L2 with free-space distances D1, D2, and D3. Specifically, the system shown in FIG. 1A includes a first light source providing visible light (identified as visible probe); a second light source providing infrared light (identified as IR source); a polarizer disposed below the first light source, the polarizer is an optical filter configured to pass light of one polarization pass through while blocking light having other polarization, thus establishing a first light path; a quarter wave plate (identified as QWP) which is configured to convert linearly polarized light to a circularly polarized light and similarly to convert a circularly polarized light to a linearly polarized light; a mirror configured to change direction of the infrared light incident onto a sample; an objective (denoted as L₁) configured to focus the combination of light from the first source and the second source onto the sample, along the first optical path; the sample disposed on a substrate (identified as sample (S)) disposed below the first focusing lens; a focusing lens (denoted as L₂) disposed in a second optical path along the polarizer that is perpendicular to the first optical path and configured to focus light exiting the polarizer along the second optical path; and a detector which is configured to receive back-reflection from the sample. The sample and substrate (denoted as Sample (S)) may include one or more thin films, made of, e.g., a thin silver film, a thin polymer film, etc., between the sample and the substrate. FIG. 1B provides a depiction of axial defocusing on the detected signal, which scales with D2 (distance between objective, L₁, and the sample) and D3 (distance between lens, L2, and the detector) on back-reflected light rays. The finite size of the detector serves as a confocal aperture. The depicted beam-path is designed to detect the integrated back-reflected beam using a polarizer/quarter wave plate/sample configuration, in which the inverted sense of circularity upon reflection results in the returning back-reflected beam being rejected at the polarizer and isolated for detection. Replacing the polarizer/quarter wave plate with a partial beam-splitter (e.g., 30% R/70% T) would provide a qualitatively similar results, but with an associated loss in detected intensity.

The goal is to model the back-reflection with changes in temperature. In a back-reflection configuration (e.g., using a polarizer, quarter-wave plate, objective design or integrating a partially reflective beam-splitter), the detected photothermal modulation in intensity can be modeled by consideration of changes in back-reflectivity for a thin sample layer on a semi-infinite substrate. In this limit, the amplitude of the modulated intensity measured by the detector in back-reflection is proportional to both the intensity of the back-reflection overall and the fractional change in that intensity with a change in temperature within the interfacial layer, as described by Eq. (1), below.

$$ΔI ∝ {I}_{R} \cdot \frac{{dI}_{R}}{dT} \qquad ( 1 )$$

where ΔI is the amplitude of the modulated intensity measured by the detector in back-reflection,
I_{R} is the intensity of the back-reflection overall, and
Tis temperature.

A more detailed discussion of the key factors affecting photothermal amplitude is provided next. For an assumed object with spatially varying thickness given by X and a concentration by C, the energy deposited in a given position will be proportional to the product of X and C evaluated at that location. In the limit of a thin sample and low IR modulation frequencies (i.e., for an rms thermal diffusion length over the modulation period much less than the sample thickness), thermal equilibrium between the sample and the substrate can be expected, such that the interfacial layer can be treated as being locally isothermal. In this framework, the IR absorption-induced temperature change for both the sample and the substrate can be assumed to be equal and proportional to the path length X and the concentration C, as provide below in Eq. (1S).

$$ΔT ∝ {σ}_{IR} {C}_{p} {I}_{IR} CX \qquad ( 1 S )$$

where σ_{IR} is . . . ,
I_{IR} is the intensity of the IR light,
Cₚ is . . . ,
X is . . . , and
C is concentration of sample.

In Eq. (1S), it is assumed that the measurements are made at sufficiently low modulation frequency and/or long time-frame such that local temperature equilibration across the interface arises. This assumption is consistent with the experimental results presented herein, in which the sample thickness was approximately 1 μm and the IR modulation period 10 μs. A rate-limiting thermal diffusivity for the bacterial colonies approximately equal to that of polyamides or water (110-140 nm²/ns) yields an rms diffusion length of about 1.5-1.7 μm, which was greater the typical maximum sample thickness (about 1 μm). As such, heat from the top-most portion of the sample is still expected to diffuse to the substrate plane over the IR modulation period.

The photothermal modulation in the back reflectance can be evaluated by the change in the amplitude reflectivity rₜₒₜ with change in temperature T.

$$\frac{{\vert Δ {r}_{t o t} \vert}^{2}}{\vert Δ T \vert} ≅ \frac{{\vert {r}_{t o t} ( {T}_{0 +} ΔT \vert}^{2} - {\vert {r}_{t o t} ( {T}_{0} ) \vert}^{2}}{Δ T} \qquad ( 2 S )$$

where rₜₒₜ is the total reflected and transmitted light emanating from the sample,
ΔT is change in temperature experienced by the sample, and
T₀ is the initial temperature of the sample. The modulation amplitude in the detected back-reflected probe beam intensity is given by the product of the reflectance sensitivity given in Eq. (2S), the incident visible probe beam intensity, and the average reflectivity. The last two terms define the average probe beam intensity at the detector, with which the modulation amplitude scales proportionally. From this product, the amplitude of the photothermal modulation measured at the detector A_{AC} is given by the following expression.

$${A}_{A C} = {I}_{v i s} ΔT {\vert {r}_{tot} \vert}^{2} \frac{{Δ \vert {r}_{t o t} \vert}^{2}}{Δ T} ∝ {I}_{v i s} {I}_{IR} {C}_{P} {ε}_{IR} CX \frac{Δ {\vert {r}_{t o t} \vert}^{2}}{Δ T} \qquad ( 3 S )$$

where A_{AC} is the amplitude of the photothermal modulation,
Iᵥᵢₛ is . . . , and
ε_{IR} is . . . .

From Eq. (3S), the photothermal response scales with the sample absorbance (proportional to the molar absorptivity SIR, the concentration C and the pathlength X), the local heat capacity the incident intensities of the visible and IR beam, the time-averaged reflectance of the interface, and the change in reflectance for the thin film/substrate system with change in temperature. The following sections are dedicated to modeling that final term.

As a first step, the following exposition is centered on determination of the back-reflected intensity as a function of film thickness and refractive index when positioned over IR transparent substrates such as CaF₂ commonly employed in O-PTIR. From Eq. (1), it is useful to consider first the factors impacting the overall reflectivity for a bare interface that collectively dictate the point-spread function (PSF), then next the thin film architecture in the immediate vicinity of the sample.

From Eq. (1), the modulation amplitude scales linearly with the back-reflected light traversing the path to the detector. As such, the detected amplitude as a function of axial and lateral position of a point photothermal source can be highly dependent on the specifics of the optical path under consideration.

The lateral resolution in a Pol/QWP/Sample optical design from a thin absorbing film is matched to the intrinsic beam-waist at the focal volume. For refractive optics the lateral point spread function will typically be described by Gaussian, Airy, or Bessel functions, depending on the objective characteristics and optical path. For refractive optics with a Gaussian far-field beam profile that fall within the aperture of the objective, the PSF in the field of view will similarly be Gaussian. A broader Gaussian in the back aperture of the objective produces a correspondingly narrow Gaussian in the field of view. As the far-field Gaussian is expanded to overfill the aperture, the aperture itself will help define the shape of the PSF. In the limit of a large degree of over-filling, the transmitted beam will adopt a “top-hat” beam profile at the back aperture, which will produce an Airy function in the field of view upon spatial Fourier transformation. Finally, when using reflective (Cassegrain) objectives such as used in this study for IR and visible compatibility, obscuration of the central component of the incident beam results in the introduction of some Bessel-beam like characteristics. These introduce nodal features in the radial and axial point spread function, along with a narrower beam waist and longer depth of field relative to a Gaussian beam with otherwise similar characteristics. In general, a combination of all three effects may contribute, leading to analytically challenging functional forms for the PSF. Fortunately, the photothermal response is anticipated to scale directly with the intrinsic lateral beam waist defined by the PSF of the incident visible probe beam for photothermal microscopy in a Pol/QWP/Sample configuration, which is often fairly straightforward to determine experimentally.

The axial resolution has the potential to be highly dependent on both the optical design for the return path and the detector characteristics. In order to characterize the axial resolution as a function of beam-path design, a ray-transfer matrix approach was adopted, the details of which are summarized below.

Two limiting cases were considered for modeling the axial point-spread function of the O-PTIR response. In the first, the sample was assumed to dominate the O-PTIR axial PSF. In this mechanism, the photothermal perturbation to the epi-detected probe beam is assumed to be proportional to the overlap between the probe beam intensity and the IR-absorbing sample, consistent with a mechanism dominated by photothermal perturbations to the incoherent back-scattered light. In this case, the 3D PSF can be used to predict the axial profile of the back-scattered intensity, which will be given by the convolution of the axial PSF with the thickness of the sample.

Calculations for both Airy and Gaussian PSF functions were performed, producing top-hat and Gaussian far-field beam profiles, respectively. In practice, the measured PSF will be more interesting than either of these two classic examples. The reflective objective produces a substantial obscuration, removing the central low-angle portion of the probe beam. Consequently, the sample is illuminated by a relatively narrow range of incident angles. In the case of the NA=0.78 reflective objective used in these studies with a transmission of 60%, the lower angle cut-off corresponds to an NA=0.49, corresponding to upper and lower angle bounds of 80=0.90 (52°) and 0.51 (29°), respectively, and an average incident angle of 0.72 (41°) for a top-hat profile.

Calculations for both Airy and Gaussian PSF functions were performed, producing top-hat and Gaussian far-field beam profiles, respectively. In practice, the measured PSF will be more interesting than either of these two classic examples. The reflective objective produces a substantial obscuration, removing the central low-angle portion of the probe beam. Consequently, the sample is illuminated by a relatively narrow range of incident angles. In the case of the NA=0.78 reflective objective used in these studies with a transmission of 60%, the lower angle cut-off corresponds to an NA=0.49, corresponding to upper and lower angle bounds of 80=0.90 (52°) and 0.51 (29°), respectively, and an average incident angle of 0.72 (41°) for a top-hat profile. In the second mechanism, it was assumed that the axial PSF of the O-PTIR response was dominated by the coherent back-reflection from the substrate. In this limit, the axial PSF can be evaluated by tracking the coherent back-reflection using ray-transfer matrices to predict the intensity roll-off as the reflecting surface is scanned through the focal plane of the objective. The general architecture for ray transfer matrix operations is depicted in Eq. (4S), in which r describes the lateral offset of a ray relative to the optical axis and θ represents the ray angle, with positive θ for a positive r corresponding to divergence of a beam and negative θ for a positive r corresponding to convergence.

$${V}_{out} = [ \begin{matrix}{r}_{out} \\ {θ}_{out}\end{matrix} ] = [ \begin{matrix}{M}_{00} & {M}_{01} \\ {M}_{10} & {M}_{11}\end{matrix} ] [ \begin{matrix}{r}_{in} \\ {θ}_{in}\end{matrix} ] \qquad ( 4 S )$$

Ray transfer matrices given by M are used to represent optical components and free-space propagation. The matrix for free-space propagation Mfs over a distance dis given by the following.

$${M}_{fs} = [ \begin{matrix}1 & d \\ 0 & 1\end{matrix} ] \qquad ( 5 S )$$

Similarly, the matrix for a thin lens Mₗ with a focal length of f is given by the following.

$${M}_{l} = [ \begin{matrix}1 & 0 \\ - 1 / f & 1\end{matrix} ] \qquad ( 6 S )$$

For an Airy function, Fourier transformation to a far-field “top-hat” profile produces a hard cut-off defined by the objective aperture. For a collimated incident beam, the ray defined by this upper limit r0 is given by an input vector vin with r=r0 and θ=0. The detected back-reflected ray following propagation along the beam path indicated in FIG. 1 is given by the following set of ray transfer matrices.

$${V}_{out} = {M}_{fs 3} {M}_{13} {M}_{fs 1} {M}_{l , obj} {M}_{fs 2} {M}_{s} {M}_{fs 2} {M}_{l , obj} {M}_{fs 1} {V}_{i n} \qquad ( 7 S )$$

The ray transfer matrix for a thin sample on a substrate MS is described by complex-valued amplitude reflectivity coefficients, which for normal incidence are equal in amplitude and opposite in sign.

$${M}_{s} = [ \begin{matrix}{r}_{p} & 0 \\ 0 & {r}_{s}\end{matrix} ] = {r}_{p} [ \begin{matrix}1 & 0 \\ 0 & - 1\end{matrix} ] \qquad ( 8 S )$$

The detected intensity for a point-source positioned along the optical axis in the object plane can be calculated from Vout evaluated in the plane of the detector according to Eq. 6. For a top-hat beam profile dictated by the objective aperture, this evaluation is relatively straightforward. Assuming a spherically symmetric detector with a radius r_{d}, the fraction of light recovered at the detector F is given by the following expression.

$$F ( {r}_{o u t} ) = \{ \begin{matrix}1 ; {r}_{out} < {r}_{d} \\ {( \frac{{r}_{d}}{{r}_{out}} )}^{2} ; {r}_{out} \geq {r}_{d}\end{matrix} \qquad ( 9 S )$$

The expression in Eq. (9S) is generated from the two-dimensional (2D) projection of the top-hat profile of the beam onto the top-hat profile of the detector. For a top-hat beam profile, the hard cut-off of the back-projected beam either falls completely within the detector area (for which all the back-reflected light is detected) or overfills the detector area, for which the fraction falling within the detector scales quadratically with the ratio of the two radii.

For a Gaussian beam, the fraction of recovered light is given by integration over a 2D Gaussian function of the projected beam from 0 to r_{d} in the radial dimension. For a ray describing a Gaussian function with a characteristic distance constant of r₀, the fraction of intensity landing within the cross-section of the detector is given by the following integral expression.

The relations in Eqs. (8S) and (9S) can be used to evaluate the axial point-spread function for a Gaussian beam profile, as they define the degree to which the signal is reduced upon translation of the object plane away from the focal plane of the objective. The expression in Eq. (9S) holds equally well for a single Gaussian function as shown, as well as for each of the Gaussian functions describing a point spread function given by a sum of Gaussian distributions.

To simulate the axial point spread function (PSF) for a beam-path design consistent with optical contrast in O-PTIR microscopy dominated by the substrate reflectivity, the active area of the detector was allowed to be an adjustable parameter in a least-squares fit to the experimental axial profile. The simulations in FIG. 1C, which provides simulations and measurement of the axial point-spread function for a beam-path design consistent with FIG. 1A and FIG. 1B, and which illustrate the relationship between the detector active area and the axial resolution in the PSF. Accordingly, two calculations were performed: i) one in which the axial PSF of the incident beam convolved with the sample dominates the photothermal activity (shown as incoherent sample-dominated), and ii) one in which the substrate reflectivity drives the axial response (shown as coherent reflection-dominated). Maximum likelihood fits to the experimental data (shown as dots) are shown for each of the two mechanisms. In brief, the physical size of the detector serves as an effective confocal aperture. Increasing the size results in corresponding increases in the depth of field. Conversely, a smaller detector area limits the collection of coherently back-reflected light, thereby narrowing the PSF.

Experimental data of a z-scan of Synechocystis bacterial colonies on a calcium fluoride substrate shown in FIG. 1C along with direct comparisons between the maximum likelihood fits of the experimental data to each of the two mechanisms (incoherent back-scattering shown in red, and coherent back-reflection shown in blue). For the back-scattering mechanism, a three-parameter fit was performed to recover most probable values for the sample thickness (1.0 μm±0.3 μm), the zero-position of the sample (−0.3 μm±0.4 μm), and the overall amplitude (3.3±0.5). Analogous fits of the data to the model for coherent back-reflection are shown in blue in FIG. 1C produced best-fit values for the detector/pinhole area (28 μm±1 μm), the zero-position (−0.51 μm±0.05 μm) and the overall amplitude (12.3±0.5).

From inspection of FIG. 1C, experimental measurements of the axial PSF for O-PTIR were in excellent agreement with the coherent back-reflection model predictions for the photothermal response, and deviated significantly from predictions based on the incident PSF. These results further support the dominance of photothermal perturbation to the coherent back-reflection arising from interfacial multilayer assemblies, and not perturbations in the direct back-reflected scatter or absorption by the sample itself. The recovered value for the detector aperture (about 30 μm when assuming a 100 mm tube length) is reasonable.

A detailed discussion of the PSF incorporating the central obscuration of the objective to produce Bessel-like illumination is provided below. The 3D point spread function adjacent to the focal plane was integrated into the modeling of optical interference effects. To evaluate the PSF adjacent to the focal plane, both the visible probe beam profile in the pupil plane and the intrinsic obscuration of the Cassegrain objective were taken into consideration. The inner NA of the obscuration was calculated based on the outer NA together with the total transmittance of the objective. The beam profile of the incident visible probe beam at the back aperture of the objective was assumed to be Gaussian, consistent with experimental observations. The obscuration was modeled by addition of another Gaussian of smaller diameter, the summation of which produced a node at low angles. A depiction of both the modeled beam profile and an overlay of the acceptance aperture of the objective are shown in FIGS. 1A-1B of the present disclosure.

Modeling the obscuration by a single Gaussian peak with negative amplitude does not capture the sharp edges in the optical transmission but nevertheless recovers the core elements of the objective spatial frequency bandpass. Furthermore, the 3D PSF of a Gaussian function is analytically straightforward to evaluate, facilitating computational modeling. Including additional Gaussian functions can reproduce the edges of the obscuration with arbitrary precision. In principle, the objective PSF could be similarly modeled by a summation over Bessel functions, each of which describes ring illumination at a particular spatial frequency vo.

Summation or integration over many such properly scaled rings can recover the measured PSF. However, the relatively low obscuration of the objective (about 40%) would require addition of many such functions to accurately recover the frequency bandpass of the objective, complicating intuitive interpretation of the sum.

$$H ( \overline{v} ) ≅ {G}_{1} ( \overline{v} - {G}_{1} ( \overline{v} ) \qquad ( 10 S )$$

Fourier-transformation of the Gaussian functions in the spatial-frequency domain at the plane of the objective (H) produced connected Gaussian functions in real-space adjacent to the focal plane (h). The real-spatial amplitude of the nth frequency-domain Gaussian function was scaled by its corresponding frequency-dependent width upon Fourier transformation.

$${h}_{n} ( r , z ) = {A}_{n} \frac{{w}_{0}}{{σ}_{n} ( z )} exp [ - \frac{{r}^{2}}{2 {σ}_{n}^{2}} ] exp [ - i ( \frac{k r}{2 R} - atan \frac{z}{{z}_{R}} ) ] \qquad ( 11 S )$$

In the preceding expression (11S), the absolute phase term exp(−ikz) corresponding to plane-wave propagation has been omitted to better emphasize the shifts relative to that reference. The definitions of several constants used in Eq. 11S), including the beam waist w₀, the Rayleigh range zR defining the depth of field, and the magnitude of the wavevector k, are given below.

$${w}_{0} = \frac{λ}{π NA} \qquad ( 12 S )$$

$${z}_{R} = \frac{π {w}_{0}^{2}}{λ} \qquad ( 13 S )$$

$${σ}^{2} = {w}_{0}^{2} [ 1 + {( \frac{z}{{z}_{R}} )}^{2} ] \qquad ( 14 S )$$

$$R = z [ 1 + {( \frac{{z}_{R}}{z} )}^{2} ] \qquad ( 15 S )$$

$$k = \frac{2 π n}{λ} \qquad ( 16 S )$$

The amplitudes of the two Gaussian functions in the spatial frequency domain are equal in peak amplitude and opposite in sign. The corresponding amplitudes in the spatial domain are scaled by the frequency-domain Gaussian widths defined by the objective.

Consistent with the curves shown in FIGS. 1D, 1E, 1F, and 1G, which provide a summary of modeling calculations for the z-dependent PSF produced by the Cassegrain reflective objective, where the frequency-domain Gaussian widths were selected to be ⅔ of the corresponding band edges (e.g., a positive Gaussian function with a width of σ_{v̅}=2×0.78/3 and a negative Gaussian with a width of σ_{v̅}=2×0.45/3). In particular, FIG. 1D provides a comparison of the objective bandpass with the approximation from the sum of two Gaussian functions, FIG. 1E provides the corresponding axial PSF of the beam produced upon spatial Fourier transformation, FIG. 1F provides representative images of the PSF at the focal plane, and FIG. 1G provides offset by 3 μm from the focal plane.

Incorporation of the frequency-domain widths in the amplitudes of the corresponding real-space Gaussian functions yields the following expressions for the real-space amplitudes.

$${A}_{n} = {( - 1 )}^{n + 1} {σ}_{\overline{v}} \qquad ( 17 S )$$

In Eq. (17S), n=1 for the larger positive Gaussian function in FIG. 1D and n=2 for the narrower negative Gaussian function.

From this summation, the full 3D PSF produced by the Cassegrain objective can be calculated, representative slices of which are shown in FIGS. 1F-1G depict the anticipated radial beam shape at select axial z-planes, including the focal plane in C., and a plane 3 μm out of focus in FIG. 1F. The radial nodes evident in FIGS. 1F and 1G can be intuited by the sum of two Gaussian beams. The broader Gaussian function depicted in FIG. 1D. with a higher integrated amplitude will produce a relatively narrow Gaussian in the focal plane. Conversely, the narrower Gaussian in A. will produce a broader and oppositely signed Gaussian in B. Summation of the two will result in sign inversion at the nodal ring, beyond which the PSF is negatively signed and dominated by the broader real-space Gaussian (and narrower at the objective).

The appearance of axial nodal minima in amplitude along the z-axis depicted in FIG. 1E can be rationalized by analogous arguments. In this particular case, in which the Cassegrain bandpass is modeled by a summation of two Gaussian functions, the nodal planes can be intuitively understood from the combination of just these two Gaussian functions. At the focal plane, the higher NA of the broader Gaussian function dominates the total amplitude. Further from the focal plane, the higher NA Gaussian function decays more quickly, with the nodal plane corresponding to the z-position for which the two Gaussian amplitudes are equal and opposite.

Of greatest practical relevance is the prediction of axial nodal planes of low optical amplitude above and below the focal plane. In the modeling, these nodal planes are recovered by simulations with the addition of a Gaussian obscuration of the central portion of the beam in the spatial frequency domain at the objective plane.

The relatively narrow incident angle range centered about 41° inherent in the reflective (Cassegrain) objective used in this study makes O-PTIR measurements performed with this objective more prone to optical interference effects than a conventional refractive objective, particularly when coupled to a narrow-band probe beam from a continuous-wave laser source. Such interferences have the potential to complicate interpretation of the corresponding images but also provide opportunities for mining additional information content on the sample.

FIG. 1H is a block diagram of the optical circuit shown in FIG. 1a. Namely, light source 1 and 2 (with light source 2 having modulation) are processed through optical components and provided to the sample on a substrate. The back-reflected light from the sample is processed through optical components and provided to the detector which includes a lock-in-amplifier to thereby output the total back-reflected light.

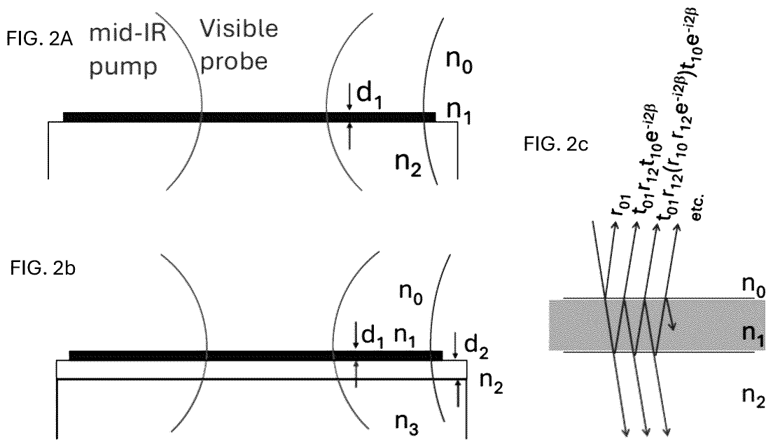

Within the limits of the thin film model for measurements integrating over the total back-reflectance or transmittance of the probe beam configuration, the PSF is anticipated to be largely decoupled from the local interactions driving the amplitude response. If the physical extent of the sample is comparable or larger than the probe beam waist, the sample can be modeled as locally planar, justifying the use of plane-wave optics for a stratified layer architecture. A depiction of this architecture is shown in FIGS. 2A-2C, which provide a thin layer model for photothermal IR reflectometry. In particular, FIG. 2A depicts a one layer model on a substrate, FIG. 2B provides a two or more layer model, and FIG. 2C provides an illustration of the collective reflection, transmission, and phase shifts driving the complex-valued reflectivity in a one-layer system.

The axial resolution in a one-layer thin-film model is determined by the interplay of the incident light's wavelength, the refractive indices of the layer, and the thin-film thickness. The dependence of the polarization state and the amplitude of the signal on the refractive index of the thin layer can be systematically analyzed by linearizing the nonlinear relationship described in Eq. 9 with the expressions for the polarization and QWP matrices at set angles given in Eq. 10 and 11. The expression for rₜₒₜ describes the total reflection coefficient for a multilayer system, accounting for multiple reflections within the thin film and considering contributions from the interface reflection coefficients, transmission coefficients, as well as the phase shifts incurred due to the optical path length within the thin layer. Given the incident angle of about 41°, both the s- and polarized components of the reflectivity should be accounted in the analysis. Expressions for the amplitude reflection and transmission coefficients are well-established.

$${r}_{tot} = {r}_{01} + {t}_{01} {e}^{- i β} {r}_{12} {e}^{- i β} {t}_{10} + {t}_{01} {e}^{- i β} {r}_{12} {e}^{- i β} {r}_{10} {e}^{- i β} {r}_{12} {e}^{- i β} {t}_{01} + {t}_{01} {e}^{- i β} {r}_{12} {{e}^{- i β} ( {r}_{10} {e}^{- i β} {r}_{12} {e}^{- i β} )}^{2} {t}_{10} + … = {r}_{01} + t {t}_{01} {r}_{12} {t}_{10} {e}^{- i 2 β} [ 1 + ( {r}_{10} {r}_{12} {e}^{- i 2 β} ) + {( {r}_{10} {r}_{12} {e}^{- i 2 β} )}^{2} + {( {r}_{10} {r}_{12} {e}^{- i 2 β} )}^{3} + … ] = {r}_{01} + \frac{{t}_{01} {r}_{12} {t}_{10} {e}^{- i 2 β}}{1 - {r}_{10} {r}_{12} {e}^{- i 2 β}} \qquad ( 2 )$$

Given the incident angle of about 41° C., both the s- and p-polarized components of the reflectivity should be accounted in the analysis. Expressions for the amplitude reflection and transmission coefficients are well-established.

$${r}_{s , ti} = \frac{{n}_{i} cos {θ}_{i} - {n}_{t} cos {θ}_{t}}{{n}_{i} cos {θ}_{i} + {n}_{t} cos {θ}_{t}} {r}_{p , ti} = \frac{{n}_{t} cos {θ}_{i} - {n}_{i} cos {θ}_{t}}{{n}_{t} cos {θ}_{i} + {n}_{i} cos {θ}_{t}} \qquad ( 3 )$$

The corresponding amplitude transmission coefficients are given by the following.

$${t}_{s , ti} = \frac{2 {n}_{i} cos {θ}_{i}}{{n}_{i} cos {θ}_{i} + {n}_{t} cos {θ}_{t}} {t}_{p , ti} = \frac{2 {n}_{i} cos {θ}_{i}}{{n}_{t} cos {θ}_{i} + {n}_{i} cos {θ}_{t}} \qquad ( 4 )$$

For a one-layer thin film, the phase-shift β₁ upon a single-pass through the thin layer (medium 1) is given by the following expression.

$${β}_{1} = \frac{2 π {d}_{1} {n}_{1} cos {θ}_{1}}{λ} \qquad ( 5 )$$

The Jones vector describing the reflected field can be generated from the Jones matrix describing the amplitude and phase of each polarization component.

$${[ \begin{matrix}{e}_{p} \\ {e}_{s}\end{matrix} ]}_{r} = {[ \begin{matrix}{r}_{p 01} + \frac{{t}_{p 01} {r}_{p 12} {t}_{p 10} {e}^{- i 2 β}}{1 - {r}_{p 10} {r}_{p 12} {e}^{- i 2 β}} & 0 \\ 0 & {r}_{s 01} + \frac{{t}_{s 01} {r}_{s 12} {t}_{s 10} {e}^{- i 2 β}}{1 - {r}_{s 10} {r}_{s 12} {e}^{- i 2 β}}\end{matrix} ] [ \begin{matrix}{e}_{p} \\ {e}_{s}\end{matrix} ]}_{in} \qquad ( 6 )$$

When using linearly polarized incident light, the relative contributions of p-polarized and s-polarized light depend on the azimuthal angle φ(eₚ−e₀ cos φ, eₛ=e₀ sin φ). However, the use of circularly polarized incident light consistent with the polarizer/QWP/objective beam path considered herein removes the azimuthal dependence and considerably simplifies the analysis. The dependence of the reflected intensity on refractive index variations can be mathematically described by the squared magnitude of the two orthogonally polarized complex amplitude reflectivities, shown in Eq. (7).

$${I}_{r} = {I}_{p} + {I}_{s} ∝ \frac{1}{2} {\vert {r}_{p 01} + \frac{{t}_{p 01} {r}_{p 12} {t}_{p 10} {e}^{- i 2 β}}{1 - {r}_{p 10} {r}_{p 12} {e}^{- i 2 β}} \vert}^{2} {I}_{in} + \frac{1}{2} {\vert {r}_{s 01} + \frac{{t}_{s 01} {r}_{s 12} {t}_{s 10} {e}^{- i 2 β}}{1 - {r}_{s 10} {r}_{s 12} {e}^{- i 2 β}} \vert}^{2} {I}_{in} \qquad ( 7 )$$

This framework ultimately connects the refractive index variations, intensity modulation, and thermal effects, to allow for quantitative analysis of sensitivity in optical systems. In particular, it has found relevance for designing thermally responsive optical devices and probing the material properties of thin films and coatings.

For an interfacial architecture containing two or more interfacial layers as illustrated in FIG. 2B., the preceding approach based on propagation of Jones vectors rapidly leads to unwieldy analytical expressions. Fortunately, complementary matrix methods employed in the design of optical thin films coatings for mirrors and polarizers can be brought to bear to track multilayer reflectivity in amplitude and phase. The mathematical framework for multilayer stacks utilizes matrix operations for each s- and p-polarization component separately. One can define a matrix S to describe the reflectivity for a given polarization state from a multilayer assembly.

$$S = {I}_{01} {L}_{1} {L}_{12} {L}_{2} {I}_{23} … \qquad ( 8 )$$

In Eq. (8), the expressions for Ij and Lij are given below.

$${I}_{i j} = ( \frac{1}{{t}_{i j}} ) [ \begin{matrix}1 & {r}_{i j} \\ {r}_{i j} & 1\end{matrix} ] \qquad ( 9 )$$

$${L}_{j} = [ \begin{matrix}{e}^{i {β}_{j}} & 0 \\ 0 & {e}^{- i {β}_{j}}\end{matrix} ] \qquad ( 10 )$$

For a two-layer model (indexed as 1 and 2) from an incident ambient medium 0 and a semi-infinite substrate indexed 3, the combinations of these equations lead to the following expression for the matrix S in a 2-layer model.

$$S = ( \frac{1}{{t}_{01} {t}_{12} {t}_{23}} ) [ \begin{matrix}1 & {r}_{01} \\ {r}_{01} & 1\end{matrix} ] [ \begin{matrix}{e}^{i {β}_{1}} & 0 \\ 0 & {e}^{- i {β}_{1}}\end{matrix} ] [ \begin{matrix}1 & {r}_{12} \\ {r}_{12} & 1\end{matrix} ] [ \begin{matrix}{e}^{i {β}_{2}} & 0 \\ 0 & {e}^{- i {β}_{2}}\end{matrix} ] [ \begin{matrix}1 & {r}_{23} \\ {r}_{23} & 1\end{matrix} ] \qquad ( 11 )$$

Extension to include more than two-layers involves the straightforward multiplication by an additional pair of matrices from Eqns. (9) and (10) for each additional layer in the multilayer stack. From the final 2×2 matrix S, the complex amplitude reflectivity rₜₒₜ and transmissivity tₜₒₜ are calculated from the following equations.

$${r}_{tot} = \frac{{s}_{21}}{{s}_{11}} \qquad ( 12 )$$

$${t}_{tot} = \frac{1}{{s}_{11}} \qquad ( 13 )$$

The expression in Eq. (12) was used to evaluate the reflectivity as a function of the sample and overlayer dielectric properties.

When evaluated for a single-layer structure, the expression for a multilayer thin film stack in Eq. (12) evaluated for either the rₛ or rₚ polarization components exactly recovers the analogous total complex reflectivity expression in Eq. (2), consistent with expectations. However, the approach depicted in Eq. (11) has the additional advantage of being directly extendible to film structures with arbitrary layer numbers.

The preceding two sub-sections allow determination of the reflectivity in a multilayer assembly as a function of the film thickness(es) and optical constants of the incident medium, the substrate, and the interfacial layer(s). Connecting these reflectivities to the optical photothermal response requires consideration of how each of the parameters impacting reflectivity change with input energy into the surrounding bath following local infrared light absorption by the sample.

Heat capacity and thermal diffusivity play critical roles in determining the local temperature change resulting from heat dissipation during and following infrared light absorption. For thin samples (< about 1 μm) in intimate contact with the substrate, thermal equilibration across the film/substrate interface can be assumed to occur rapidly over the timeframe of the infrared modulation period (typically >10 μs) due to comparatively fast thermal diffusivity, effectively equalizing the temperature over the film/substrate interface. Consequently, the temperature change induced by localized heat dissipation can be considered approximately spatially uniform across the thin film on a semi-infinite substrate, provided the distances involved are short compared to the thermal diffusion length. This assumption simplifies the analysis of heat flow and temperature gradients in the modeling of temperature-dependent processes within thin film systems, albeit at the expense of complexity in modeling the impact on reflectivity.

This isothermal assumption within the focal volume may not hold for short times following IR illumination and/or for particulate samples in poor thermal contact with the substrate. In such cases, the initial temperature change induced within the sample may not have sufficient time to reach thermal equilibrium with the substrate. Under these conditions, the theoretical modeling is considerably more straightforward, requiring only consideration of the thermal/optical properties of the sample alone. This limit will not be considered in the optical modeling presented in the present disclosure, as it does not apply under the conditions in which the measurements presented were obtained experimentally. In the present disclosure, the film thickness (about 1 μm) was much less than the depth of field of the microscope with modulation periods (about 10 μs) much longer than the thermal diffusion time necessary to equilibrate across the sample/substrate interface.

Under isothermal conditions within the focal volume, the change in reflectivity of the thin film structure can be interpreted with respect to transient changes in temperature of the entire system within the focal volume. Two key factors contribute to this change with temperature: i) the refractive index of each medium at the interface through its corresponding thermo-optic coefficient, and ii) the sample thickness by the thermal expansion coefficient. The change in refractive index with temperature must be considered for both the sample and the substrate. The interplay between the two will drive the total change in reflectivity. For the thermal expansion coefficient, it is assumed that only the change in thickness within the thin film architecture will drive changes in reflectivity (or equivalently, transmissivity). In principle, sufficiently large local changes in temperature throughout the substrate could result in gross changes in the focal plane. However, such contributions are unlikely to contribute to the observed modulation given the relatively small temperature change induced in the sample and the relatively length depth of field in the reflection-based instrument design. A detailed description of the operations used to predict the photothermal response as a function of the underlying structure is provided herein.

TABLE 1
Summary of Optical Constants

| | Refractive (532 | Thermo-optic | Thermal expansion |
|---|---|---|---|
| | coefficient mm) | index (ppm) | coefficient (ppm) |
| CaF₂ | 1.43 | 0.38 | 18 |
| Silicon | 9.80 | 186 | 2.6 |
| Sample | 1.5 | −115 | 200 |

In the modeling of the O-PTIR response, values for the thermo-optic and thermal expansion coefficients for common substrates are provided in Table 1. For the thin films of transferred bacterial colonies, the optical constants, including refractive index, thermos-optic coefficient, and thermal expansion coefficient were assumed to be similar to those of organic polymers for which such optical constants have been tabulated. Values were selected to also be consistent with an empirical linear relationship between the thermo-optic and thermal expansion coefficients reported previously.

### Results and Discussion

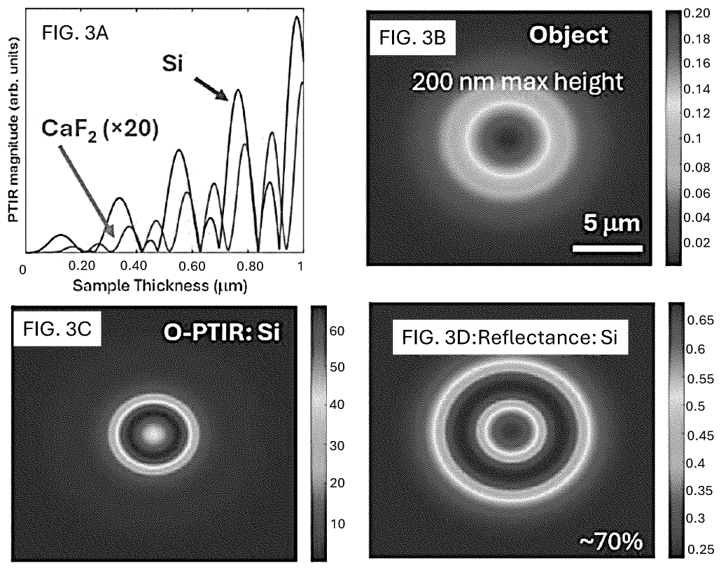

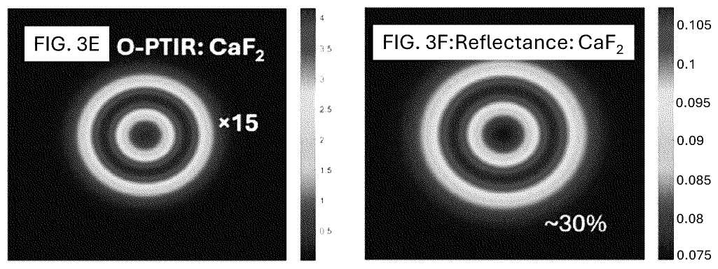

FIGS. 3A-3F provide the results of simulations of the anticipated O-PTIR responses for a sample varying in height, acquired with a beam path similar to that depicted in FIGS. 2A-2C. In particular, FIG. 3A illustrates the predicted sensitivity of a sample with a refractive index of 1.5 on either Si or CaF₂. For a sample with a Gaussian height distribution depicted in FIG. 3B, the O-PTIR and coherent reflectance responses for Si substrates are shown in FIGS. 3C and 3D, respectively. The corresponding responses for a CaF₂ substrate are shown in FIGS. 3E and 3F, respectively. As illustrated in FIG. 1 and described mathematically in Eq. (12), the total back-reflectance contains contributions from the interference from the reflections arising from the air/sample and sample/substrate interfaces, modulated by the spatially dependent phase shifts induced upon traversal across the sample. Intriguingly, the results depicted in FIG. 3A suggest about 20× enhancement in O-PTIR sensitivity for samples prepared on silicon wafers relative to those on calcium fluoride. This predicted enhancement is driven by a combination of an overall greater back-reflected intensity from the higher refractive index of Si together with a larger relative contribution from the reflection at the sample/substrate interface (r₁₂) from the larger change in refractive index across the sample/substrate interface.

The simulation results shown in FIGS. 3A-3F illustrate the interplay between the overall reflectance and the O-PTIR sensitivity. From inspection of FIG. 3D and FIG. 3F, the general patterns for the static reflectivity (i.e., not modulated by IR absorption) are identical for the two substrates, but inverted in sign. The sign change in the two interference patterns with film thickness arises from the static refractive index difference between the sample and substrate; the assumed sample refractive index of 1.50 is slightly higher than that of CaF₂ of 1.43 but lower than the refractive index of Si of 3.8. Apart from the sign change and magnitude of difference, the spatial pattern of the static reflectance is similar between the two substrates, with the maximum in one case corresponding precisely to the minimum in the other. In contrast, the maxima and minima in the anticipated photothermal response exhibit markedly different locations. In the case of Si, the maximum magnitude in the sensitivity shown in FIG. 3C is positioned along the inner edge of the primary reflectance ring shown in FIG. 3D. In contrast, the greatest sensitivity in O-PTIR on CaF₂ is predicted to arise on the outer edge of the same primary reflectance ring.

In both instances, the higher calculated O-PTIR signals on the edges of the interference fringes in the reflectance curves arise from sensitivity-enhancement from detection in quadrature. Distances close to the maxima and minima in reflectance produce only subtle changes in reflection for changes in film thickness (or equivalently, subtle changes in refractive index). However, along the edge of the fringes, subtle changes in optical constants can produce substantial changes in overall reflectivity, maximizing photothermal sensitivity.

The preferential enhancement for the inner edge of the interference fringe for samples on Si versus the outer edge for CaF₂ can be understood based on the interplay between the substrate and sample contributions. Qualitatively, the substrate produces a contribution to the detected back-reflected photothermal perturbation dictated by the sign and magnitude of the substrate thermo-optic coefficient. Depending on the sign of the thin film interference condition, this background response can either enhance or oppose the photothermal contributions from the sample. In contrast to CaF₂ with a refractive index of 1.43, the refractive index of the substrate is greater for Si than for the thin bacterial film (3.8 vs. 1.5, respectively), resulting a sign difference for the reflectance, and an enhancement in the O-PTIR amplitude along the inner edge of the reflectance ring for Si substrates and along the outer edge for CaF₂. Much of the predicted enhancements in the O-PTIR responses from samples prepared on Si substrates are attributed to precisely such interferences, driven by the comparatively large photothermal response from silicon.

In interpreting both the simulation results shown in FIGS. 3A-3F and the fitting of experimental measurements (vide infra), the role of the optical point spread function on the observables was considered in some detail. Notably, the PSF produced from reflective objectives such as employed in this study are well established to produce Bessel-like beams with correspondingly structured PSFs. Nodal content inherent in the PSF has the potential to complicate differentiation of interference effects from local changes in sample thickness versus those inherent within the PSF. A full description of the approach taken to model the PSF produced from the reflective objective is detailed herein. In brief, the PSF was modeled by assuming a Gaussian probe beam, with the central obscuration inherent in the reflective objective accounted for through summation of an additional Gaussian function with negative amplitude. Propagation of the sum to the focal plane provided straightforward analytical evaluation of the amplitude and phase of the PSF in the 3D volume adjacent to the focal plane.

All simulations and fitting were performed using this Bessel-like PSF accounting for the central obscuration of the objective used in the experiments. However, virtually identical results were recovered in both simulations and fitting when performed with a simple Gaussian PSF instead. This observation is easily rationalized; the root mean square widths of the samples investigated were >1 μm, while the PSF exhibited intrinsic features spanning distances less than about 500 nm.

Under these limits, convolution of the larger samples with the PSF produced images dominated by the features inherent in the samples and largely insensitive to the detailed structure of the PSF.

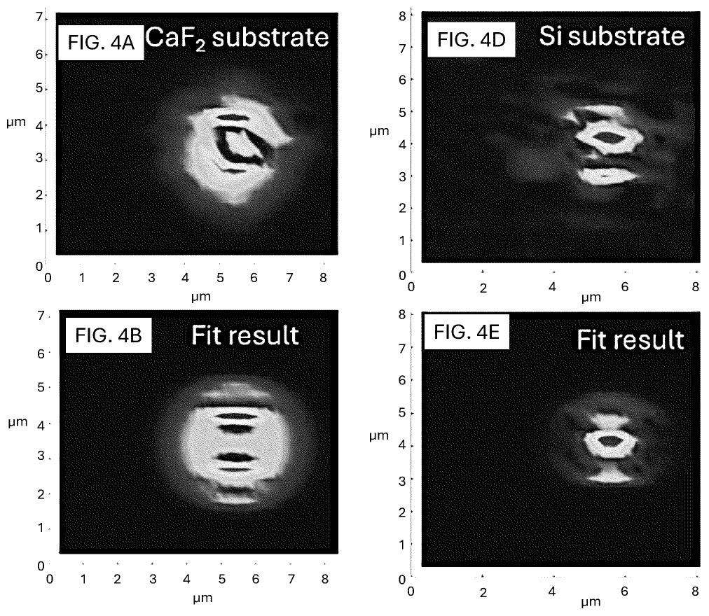

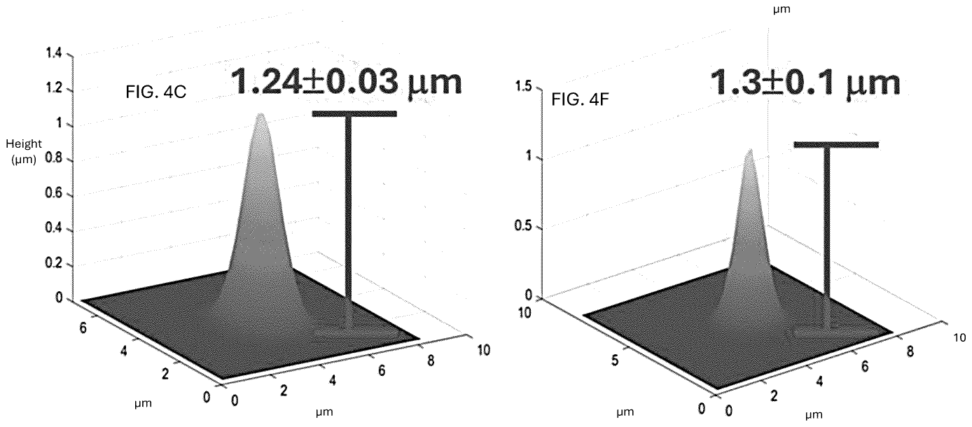

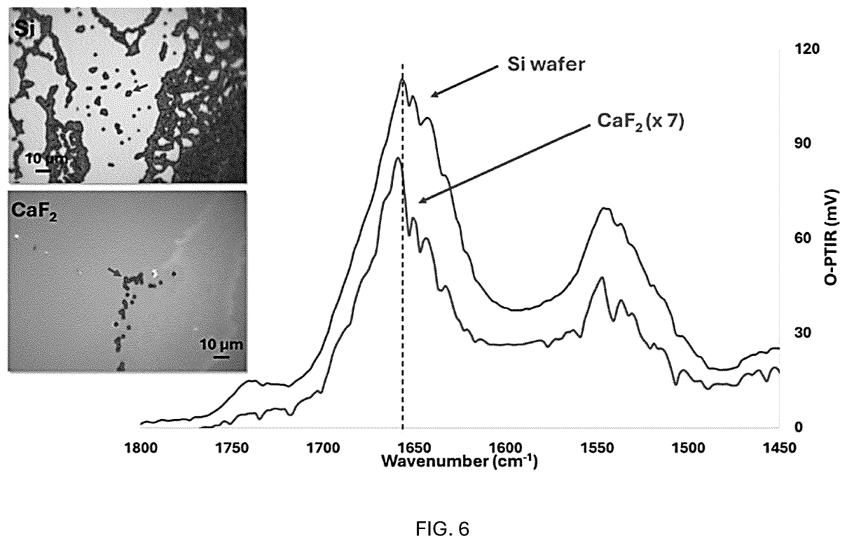

These simulated results provide a framework for interpreting differences in experimental O-PTIR optical contrast in hyperspectral microscopy measurements of thin film samples prepared on different substrate materials. A representative set of experimentally observed O-PTIR results are shown in FIGS. 4A-4F for measurements of Synechocystis prepared on CaF₂ and silicon substrates. In particular, raw experimental data are shown in FIGS. 4A and 4F with the maximum likelihood estimates of the experimental observables shown in FIGS. 4B and 4E, with the Gaussian sample height distribution shown in FIG. 4C and FIG. 4F. Calculations were performed assuming a refractive index for the bacterial film of n=1.5. Additional measurements provided in FIG. 6, which provides representative amide vibrational spectra of Synechocystis colonies on IR-transparent substrates, demonstrate the reproducibility of the trends represented in FIGS. 4A-4F. The inset in FIG. 6 displays bright-field images of the field of view (FoV), with arrows indicating the regions of interest (ROI). Measurements on the Si wafer were conducted at a lower probe power compared to CaF₂ but exhibited a higher signal-to-noise ratio (SNR). To facilitate comparison, the CaF₂ spectrum was scaled by a factor of 7. The dashed line marks the peak intensity at about 1659 cm⁻¹, where imaging and PSF evaluations were performed. These figure also includes the results of maximum likelihood at least squares fits of the experimental data to the forward model described in FIGS. 3A-3F, assuming a thin bacterial film sample producing a Gaussian distribution in heights. Overall, good agreement was observed between the predicted and observed ring-like interference patterns in the O-PTIR measurements. Similarly good agreement was observed on several similarly prepared samples.

Furthermore, maximum likelihood fitting of O-PTIR microscopy measurements of bacterial colonies transferred to calcium fluoride and to silicon substrates produced comparable values for the recovered colony heights and widths, despite producing markedly different photothermal images on the two different substrate materials.

For a given colony, the absolute thickness of the sample shown in FIGS. 4A-4F was recoverable to a precision of about ±20-100 nm on average from the fitting of the O-PTIR responses on either substrate material when incorporating optical interference effects. The precision of these results is particularly noteworthy in the studies performed on CaF₂, as the relatively subtle refractive index difference between the sample (1.5) and the substrate (1.43) results in a comparatively small back-reflection at the substrate/sample interface. While far greater accuracy in film thickness is routinely achieved in optical ellipsometry, precisions of less than 1% uncertainty in absolute sample thickness have not been reported previously in photothermal microscopy. Such precise determination of thickness supports the potential for absolute quantification of local concentrations from Beer's Law analysis. Once the path length through the sample is known precisely, the absolute concentration of an analyte can be determined unambiguously from a combination of the measured photothermal spectrum and instrument calibration with a standard.

While the results shown here center on the detected sensitivity based on the measured change in reflectivity, measurements integrating over the transmitted intensity will generally scale similarly. For transparent substrates, increases in reflectivity correspond to reductions in coherent transmissivity, and vice versa. However, reflection measurements have several practical advantages: i) reflection measurements can be performed for analysis of optically attenuating substrates, such as the use of silicon substrates with visible-light probe beams, ii) the smaller back-reflected intensity produces a larger relative modulation depth, with corresponding reductions in measurement shot-noise from the probe beam, and iii) alignment is straightforward, as the epi-detected beam passes through much of the same optical path as the incident light. However, these predictions apply exclusively to the coherent contributions to the reflection and transmission; incoherent contributions from scattering, absorption, or other dissipative loss mechanisms can also contribute significantly to attenuation in both the reflected and transmitted intensity not captured by the interferometry model presented in the present disclosure.

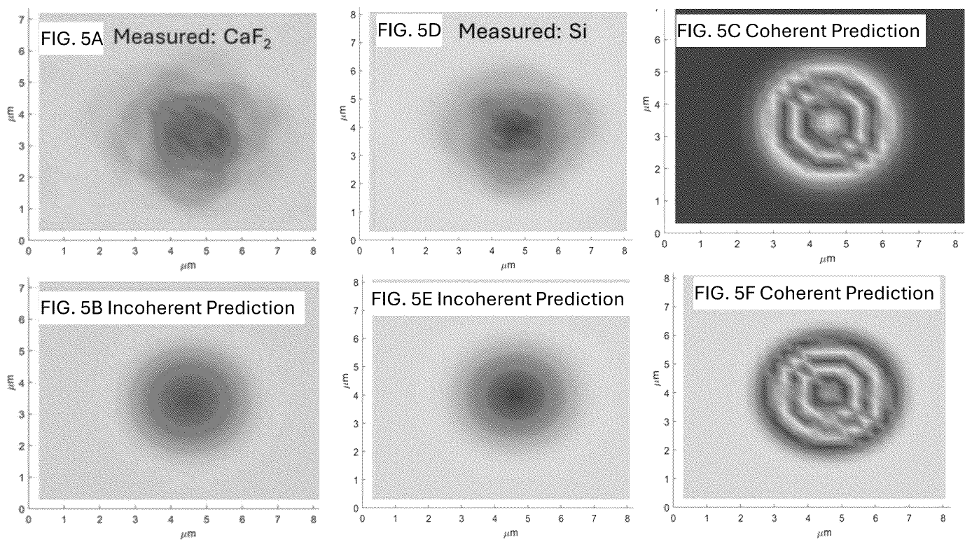

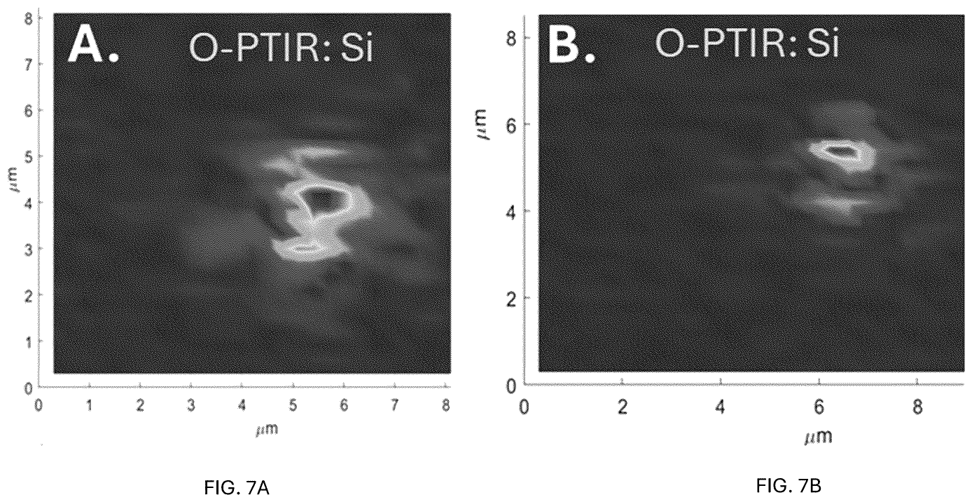

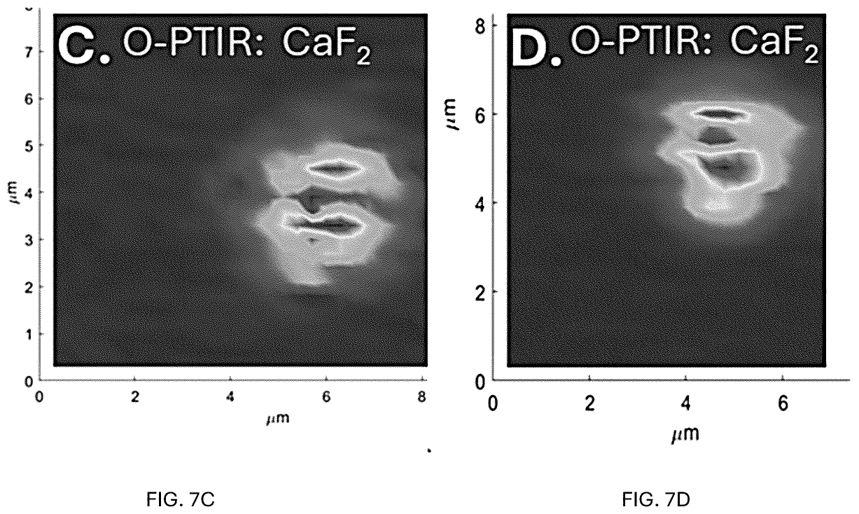

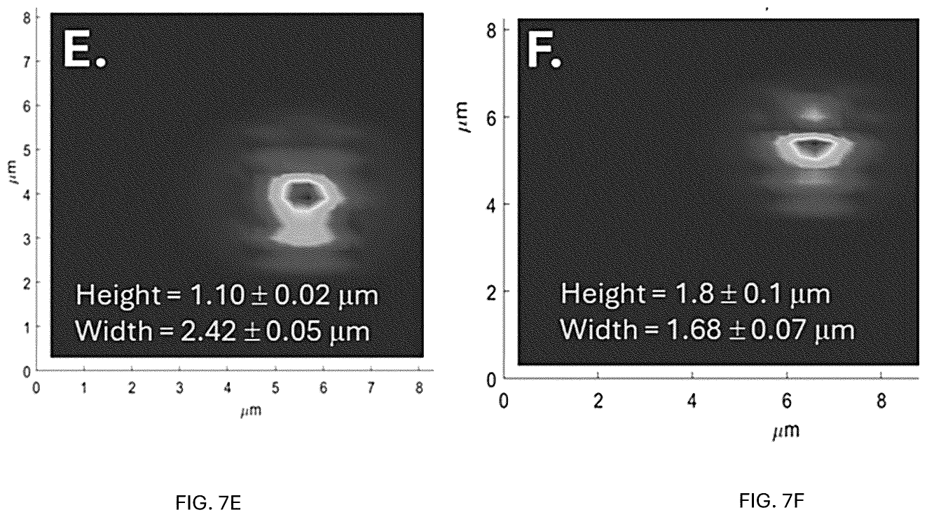

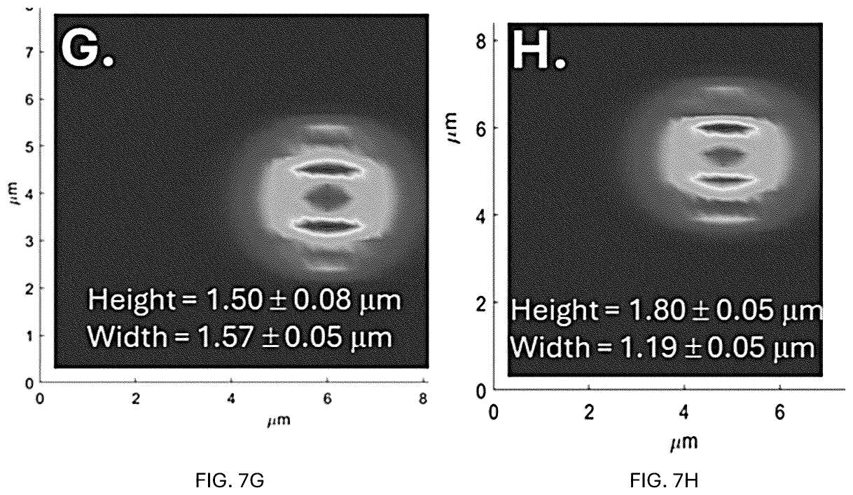

In principle, both the incoherent and coherent contribution to the modulation in back-reflection could potentially drive the measured photothermal image contrast. The relative importance of each of these two contributions was evaluated by comparison of the measured O-PTIR response with predictions based on coherent versus incoherent mechanisms, the results of which are summarized in FIGS. 4A-4F and FIGS. 5A-5F, which provide results for Time-averaged (DC) reflectance images obtained simultaneously with the O-PTIR micrographs. In particular, FIG. 5A provides measured reflectance of a bacterial colony on CaF₂ substrate with the predicted incoherent contribution to reflectance, given by the intensity of the best-fit sample height profile convolved with the PSF which is provided in FIG. 5B and predictions for coherent back-reflectance which is provided in FIG. 5C, with analogous measurements and predictions for a representative colony on an Si substate are given in FIGS. 5D, 5E, and 5F. The results in FIGS. 4A-4F are consistent with coherent interactions dominating the back-reflected O-PTIR microscopy measurements. In this limit, both the reflectance and O-PTIR results should scale identically for an isolated compositionally homogeneous sample, both of which are proportional to the underlying sample convolved with the optical point-spread function given in FIG. 5B and FIG. 5E for measurements acquired on CaF₂ and Si substrates, respectively. From inspection of the indicated panels in FIGS. 4A-4F and FIGS. 5A-5F, it should be evident that the incoherent contributions fail to adequately capture the experimentally observed O-PTIR micrographs, while the coherent contributions reasonably recover the measured patterns. These trends were reproducible across multiple analyzed samples; additional examples are included in FIGS. 7A-7H, which provide assessment of reproducibility of OPTIA analysis of O-PTIR data, for both CaF₂ and Si substrates. Measurements for the data shown presented herein on both silicon (FIG. 7A and FIG. 7B) and CaF₂ (FIG. 7C and FIG. 7D) substrates. The corresponding maximum likelihood fits based on OPTIA are presented in FIGS. 7E through 7H, respectively, with the best-fit values for the heights and widths provided. Similar growth rates of cyanobacteria result in reproducible colony size and characteristics for colonies produced by single bacteria. This reproducibility supports repeated optical characterization of similar but independent biological assemblies, which in turn can be useful for assessing the reproducibility in the measured O-PTIR data. Furthermore, the sample transfer process through physical contact with the substrate is likely to produce similar samples when prepared on CaF₂ and Si, both of which present reasonably hydrophilic interfaces. Additional representative results of several independent trials of cyanobacterial colonies are provided in FIGS. 7A-7H. Results are shown for colonies transferred to both CaF₂ and Si substrates.

The qualitative and quantitative agreement between the results shown in FIGS. 7A-7H and the representative results shown elsewhere herein attest to the reproducibility of the measurements and analysis. Qualitatively, the key nodal features and overall shape and size of the O-PTIR micrographs are in excellent agreement with the results shown in FIGS. 4A-4F. Quantitatively, the heights and widths of the colonies recovered from maximum likelihood estimates exhibited uncertainties and with reasonable biological variability well outside of experimental error. Finally, the recovered thicknesses were all on the order of the widths of individual cyanobacteria within the thin films, consistent with the transfer of single bacterial layers.

An analogous analysis can be performed to assess the relative contributions from coherent versus incoherent interactions in the time-averaged reflectance, obtained simultaneously with the O-PTIR measurements from the detector DC amplitude. The results of such a comparison are shown in FIGS. 5A-5F, again for measurements acquired on both CaF₂ and on Si substrates. The incoherent response was assumed to scale proportional with sample thickness, evaluated from the convolution of the recovered Gaussian sample height profile with the probe beam PSF. Most obviously, the incoherent contributions universally resulted in attenuation in the back-reflection relative to the bare substrate through a combination of optical scattering and probe-beam absorption by the sample. In contrast, the time-averaged back-reflectance considering only coherent optical interference effects resulted in predicted patterns exhibiting several nodal rings for both CaF₂ and Si substrates. Furthermore, the sign of the coherent contributions to the DC reflectance is predicted to invert between the two substrates, as the presumed sample refractive index of 1.5 is greater than that of CaF₂ but less than that of Si. Experimental measurements given in the top row of FIGS. 5A-5F clearly are in much better agreement with the predictions based on incoherent contributions.

The dominance of coherent contributions to O-PTIR and incoherent contributions to the time-averaged reflectivities is worthy of additional discussion. Absorption of the probe beam by the sample could potentially result in attenuation of the DC reflectance in a manner consistent with observations. Assuming an electronic molar absorptivity of about 105 L/mol-cm for a light-harvesting chromophore, a path length of about 1 μm and a cellular concentration of chromophore on the order of about 1 mM corresponds to an absorbance of about 0.01, corresponding to a about 2% reduction in reflectivity. Although the green light of the 532 nm probe beam famously sits in an optical transparency window of biological light-harvesting complexes, absorption of the probe may still be potentially non-negligible. In such cases, attenuation of the probe beam from absorptive losses could overwhelm comparatively subtle differences in reflectivity, producing reflectance profiles mirroring absorption generated by the convolution of the object with the PSF depicted in FIG. 5.B and FIG. 5.E. Optical scattering can also contribute to optical extinction and corresponding reductions in the probe reflectivity. Furthermore, optical scatter is certainly expected to arise, as it is the driving mechanism for contrast in bright-field microscopy. As with absorption, scattering will generally result in attenuation of the probe beam. If one assumes that the bacterial colonies exhibit internal spatial heterogeneity, the total scattering cross-section could easily scale with the path length through the biological film, again producing attenuation mirroring the convolution of the object with the PSF as in FIG. 5B and FIG. 5E.

The excellent quantitative agreement between the measured and predicted DC reflectivities in FIGS. 5A-5F is worthy of special note. The results shown in FIG. 5B and FIG. 5E were calculated with no additional adjustable parameters directly from the recovered best-fit height distributions of the samples via maximum likelihood fits of the images in FIGS. 4A-4F. Although the measured DC amplitudes were completely excluded from the fitting of the O-PTIR data, the predicted DC amplitude maps produced from the O-PTIR data are in excellent agreement with the measured results. This observation further supports the quantitative reliability of the optical photothermal interferometry analysis (OPTIA) approach. In addition, it also serves to highlight the unique and highly complementary nature of the image contrast in O-PTIR versus DC epi-reflectance images.

If incoherent scattering losses dominate the DC reflectivity, it is somewhat surprising that the O-PTIR signals appear to be dominated by coherent effects from interferences in reflectivity, given that O-PTIR can arise from modulation in either the coherent reflection or the incoherent scattering. In the latter case, thermally-induced changes in the scattering cross-section inform on particle absorption. Prior O-PTIR and F-PTIR studies of powdered samples have reported high sensitivity of the measurements to single-particle absorption. However, the current experimental design differs from these prior studies in two key respects. First, the sample sits in intimate contact with the substrate (relative to the thermal diffusion length). This first effect provides a thermal sink for temperature modulation in the sample, attenuating the temperature change integrated over the modulation period. As a consequence, the measured change in net reflectivity is influenced not only by the photothermal changes of the sample, but also of the substrate. Second, the back-reflected signal in the current study was detected through an effective confocal aperture dictated by the active area of the detector. As a consequence, the detected signal is dominated by the coherent response, with incoherent contributions inferred indirectly by the loss in the detected coherent back-reflections. Prior work on powders was performed by detection of modulation in the scattered incoherent signal directly, rather than by attenuation of the coherent transmission. It is reasonable to suggest that the confocal detection configuration used in these studies preferentially isolates the coherent changes in reflectivity in O-PTIR arising from interfacial interferometry, while the larger but comparatively photothermally insensitive changes in scattering and absorption dominate the sample-induced losses in DC amplitude, consistent with conventional bright-field imaging.

If the photothermal response of the substrate plays a substantial role in the overall measurement sensitivity in O-PTIR microscopy as these measurements suggest, it opens up opportunities for rational design of substrates for SNR optimization in O-PTIR. As one example, the simulations depicted in FIGS. 3A-3F predict a substantial SNR enhancement upon replacing CaF₂ slides with Si wafers as substrates for O-PTIR. The measurements shown in FIGS. 4A-4F allowed for assessment of the reliability of this prediction. To experimentally assess the influence of substrate on the signal intensity, the signal-to-noise ratio (SNR) of the two different described substrates—CaF₂ and Si—was compared. Using representative data with the highest signal for each substrate, the SNR for CaF₂ was found to be approximately 113, while the SNR for Si was around 695. After accounting for the different probe powers applied for the different substrates (20% for CaF₂ and 7.4% for Si, resulting in a 2.7-fold power differential for the probe beam), the adjusted SNR for Si increases to approximately 1880. Given the linear scaling of the signal with probe beam power, these measurements correspond to a 17-fold increase in the SNR when using a Si wafer as a substrate compared to a CaF₂ slide. This enhancement factor is in excellent quantitative agreement with the predictions illustrated in FIGS. 3A-3F, suggesting a about 20-fold improvement in the SNR of O-PTIR microscopy of thin, surface-adherent samples when switching from CaF₂ to silicon as the substrate. Rational design of multilayer substrate architectures has the potential to provide further enhancements. Furthermore, the mathematical framework described herein is intentionally designed for compatibility with multilayer thin film architectures, considerably expanding the scope of structures accessible for computational modeling and rational design.

Additionally, there is a need for modeling of the temporal blur in O-PTIR micrographs. Nominally cylindrical bacterial colonies consistently produced O-PTIR micrographs exhibiting two related characteristics: i) the O-PTIR micrographs were radially asymmetric, with lobes consistently more prominent along the vertical axis than the horizontal axis, and ii) the O-PTIR micrographs were spatially offset along the horizontal axis in the direction of the fast scan-axis relative to concurrently acquired reflectance micrographs. These combined observations are mutually consistent with O-PTIR measurements acquired with a lock-in amplifier (LIA) time-constant selected to be comparable to or larger than the single pixel dwell time. In this case, the rise/fall time of the LIA can result in significant temporal shifts in the amplifier output. In beam or sample scanning instrumentation, this temporal shift corresponds directly to a both a lateral displacement and image smoothing along the fast-scan axis.

These two effects (lateral image shift and smoothing) are illustrated in simulations and measurements shown in FIGS. 8A-8E, which provide simulated and measured influence of an extended LIA time-constant on O-PTIR image acquisition. In particular, FIG. 8A provides an illustration of the anticipated response from a single-pixel signal with a time-constant equal to three times the pixel dwell time (3×), FIG. 8B illustrates original “ground truth” O-PTIR image prior to temporal smoothing, and FIG. 8C shows resulting simulated O-PTIR image accounting for a 3× sensor exponential rise/fall time for sample-scanning from left to right, producing both a horizontal blur and lateral displacement. These results agree qualitatively with measured shifts between reflectance images such as shown in FIG. 8D and the corresponding O-PTIR images such as in FIG. 8E, both of which were acquired simultaneously from the DC and AC outputs, respectively, of the same optical sensor. The time-constant of the sensor can be modeled by convolution of the O-PTIR image with a single-sided exponential function in time, depicted in FIG. 8A for a single-pixel response along the fast-scan axis. The impact of such a filter on O-PTIR measurements is illustrated in FIGS. 8B and 8C, depicting a simulated “ground truth” O-PTIR micrograph and the same micrograph acquired assuming horizontal sample-scanning from left to right, evaluated for an exponential rise/fall time constant three times larger than the pixel dwell time.

These results are in excellent qualitative agreement with experimental observations. The lateral offset is consistent with measured lateral displacements between the reflectance and O-PTIR micrographs, both of which were obtained concurrently using the same sensor.

Representative results are shown in FIG. 8D and FIG. 8E. The O-PTIR measurements were obtained from the AC component of the signal after passage through a high-speed digital lock-in amplifier, whereas the reflectance data were generated from direct recording of the sensor output. In addition to the obvious lateral displacement of the two images along the fast-scan axis, the O-PTIR image appears to exhibit evidence of horizontal smoothing, again consistent with a significant degree of temporal smoothing.

Those having ordinary skill in the art will recognize that numerous modifications can be made to the specific implementations described above. The implementations should not be limited to the particular limitations described. Other implementations may be possible.

1. A method for determining absolute thickness of bacterial colonies, comprising:
transferring one or more bacterial colonies to an infrared (IR) transparent substrate;
along a first light path, shining visible light from a first light source;
polarizing the shone visible light along the first light path, thus polarizing the visible light at a predetermined linear polarization;
along the first light path, converting the linearly polarized light to circularly polarized light;
along the first light path, combining IR light from a second light source with the linearly polarized light;
along the first light path, focusing the combination of IR light and the linearly polarized light by an objective onto the IR transparent substrate with the one more bacterial colonies disposed thereon;
back-reflecting probe beam light from the one or more bacterial colonies along the first light path;
polarizing the back-reflected probe beam light along a second light path;
along the second light path, focusing the polarized back-reflected probe beam light by a lens;
along a second light path, receiving the polarized back-reflected probe beam light by a detector; and
performing optical photothermal mid-infrared (O-PTIR) measurements on the detected light while varying IR and back-reflected probe beam light power settings optimized for the substrate to thereby determine the absolute thickness of bacterial colonies.

2. The method of claim 1, wherein the one or more bacterial colonies are cyanobacterium Synechocystis PCC 6803.

3. The method of claim 1, wherein the step of transferring one or more bacterial colonies to the IR transparent substrate, includes:
initially placing the bacterial colonies on a transfer slide;
placing the transfer slide on the IR transparent substrate; and
applying minute pressure to cause transference from the transfer slide onto the IR transparent substrate.

4. The method of claim 3, wherein the transfer slide is made of one or both calcium fluoride and silicon.

5. The method of claim 4, wherein if the transfer slide is made of calcium fluoride, the IR power is about 22% with probe maintained at about 20% power.

6. The method of claim 4, wherein if the transfer slide is made of silicon, the IR power is about 22% with probe maintained at about 7.4% power.

7. The method of claim 2, wherein the applied IR has a spectral range of about 1450 cm⁻¹ to 1800 cm⁻¹, targeting amide peak region characteristic of the one or more bacterial colonies.

8. The method of claim 1, wherein the first optical path and the second optical path are perpendicular to one another.

9. The method of claim 1, further comprising modeling the back-reflected probe beam light with changes in temperature.

10. The method of claim 9, wherein the model is based on:
$$Δ I ∝ {I}_{R} \cdot \frac{{dI}_{R}}{d T} ,$$
Wherein ΔI is amplitude of the modulated intensity measured by the detector in the back-reflected probe beam light,
I_{R} is the intensity of the back-reflected probe beam light, and
T is temperature.
